(19) United States
(12) Patent Application Publication
(10) Pub. No.: US 2024/0049722 A1
(43) Pub. Date: Feb. 15, 2024

(54) METHOD FOR INCREASING FERROUS ION CONTENT IN BACILLUS COAGULANS AND COMPOUND MICROBIAL AGENT

(71) Applicants: Qingdao Agricultural University, Qingdao (CN); Shandong Xingu Health Industry Co., Ltd, Binzhou (CN); Qingdao Huihe Biotechnology Co., Ltd, Qingdao (CN)

(72) Inventors: Baowei WANG, Qingdao (CN); Kexin LI, Binzhou (CN); Hanyu ZOU, Qingdao (CN); Binghan WANG, Qingdao (CN); Mingai ZHANG, Qingdao (CN); Wenlei FAN, Qingdao (CN); Zhuyu YANG, Qingdao (CN); Min KONG, Qingdao (CN)

(21) Appl. No.: 18/446,057

(22) Filed: Aug. 8, 2023

(30) Foreign Application Priority Data
Aug. 11, 2022 (CN) 202210960002.X

(51) Int. Cl. A01N 63/22; C12N 1/20

(52) U.S. Cl. CPC A01N 63/22; C12N 1/20; C12N 2500/24; C12N 2523/00; C12N 2500/72; C12R 2001/07

(57) ABSTRACT

A method for increasing iron ion content in Bacillus coagulans includes following steps: simultaneously fermenting and culturing Bacillus coagulans and Clostridium butyricum in a culture medium rich in iron ions, where a concentration of the iron ions in the culture medium is 250-350.0 mg/L. Bacillus coagulans has been domesticated in the culture medium containing high concentration of iron ions, and one of them is Bacillus coagulans strain with preservation number of CGMCC No. 24223. The application also provides a compound microbial agent, which contains domesticated Bacillus coagulans and Clostridium butyricum.

### CROSS-REFERENCE TO RELATED APPLICATIONS

This application claims priority to Chinese Patent Application No. 202210960002.X, filed on Aug. 11, 2022, the entire contents of which are incorporated herein by reference.

### TECHNICAL FIELD

The application belongs to the technical field of livestock and poultry microecological preparation, and particularly relates to a method for increasing iron ion (Fe²⁺) content in Bacillus coagulans and a compound microbial agent.

### BACKGROUND

Inorganic iron and organic iron are commonly contained in feed. What animals absorb is ferrous ions (Fe²⁺). Ferrous ions (Fe²⁺) dissociated from both organic and inorganic iron entering the organism is able to be absorbed by the organism. However, ferrous ions (Fe²⁺) is easily oxidized to ferric ions (Fe³⁺) in aerobic environment, which affects the absorption of iron ions. The ferric ions (Fe³⁺) absorbed by the body is only one third of ferrous ions (Fe²⁺). The biological value of organic iron sources is higher than that of inorganic iron source.

In the field of livestock and poultry breeding, adding suitable ferrous ions (Fe²⁺) to feed is able to promote the growth and development of livestock and poultry and to enhance the quality of egg shells of egg-laying poultry. It is reported that the utilization rate of iron digestion and absorption is very low, and the iron absorption rate in livestock feed is only 10%-25%. Most of the iron directly added in the feed is discharged out of the body. Especially in large-scale livestock and poultry breeding, the content of iron ions discharged from manure into the environment is very high, which will easily lead to eutrophication of water bodies and high content in livestock and poultry products over time, thus throwing threats to food safety and the whole ecological environment. Therefore, it is of great significance for the precise nutrition of livestock and poultry breeding and the maintenance of a good ecological cycle to explore a method which is able to not only promote the normal growth and development of livestock and poultry, maintain intestinal health and improve the strength of poultry eggshells, but also reduce iron emissions is of great importance.

In the previous research done by the applicant, it is found that during the fermentation process, Bacillus coagulans is able to rapidly consume free oxygens in the external environment or animal intestines, contributing to hypoxia in the environment or animal intestines, greatly reducing the oxidation of ferrous ions (Fe²⁺) into ferric ions (Fe³⁺) in the aerobic environment, and maintaining the absorption activity of ferrous ions (Fe²⁺).

However, Bacillus coagulans, which has not been domesticated, is inert to metal enrichment to some extent, especially to Fe²⁺ enrichment, which is very limited.

### SUMMARY

The purpose of the present application is to provide a method for increasing the iron ion content in Bacillus Coagulans, thereby effectively increasing the iron ion content in Bacillus Coagulans and making up for the defects of the prior art.

The method for increasing the iron ion (Fe²⁺) content in Bacillus coagulans provided by the present application involves simultaneously fermenting and cultivating Bacillus coagulans and Clostridium butyricum in a culture medium rich in iron ions.

The concentration of iron ions in the culture medium is 250-350.0 milligrams per liter (mg/L), preferably 300.0 mg/L.

Optionally, a specific composition of the culture medium is as follows: 5-15 grams per liter (g/L) of peptone, 1-5 g/L of beef extract, 1-10 g/L of NaCl (sodium chloride), Fe²⁺ with a concentration 250-350.0 mg/L, pH of 5.5-7.9±0.2.

Optionally, the Bacillus coagulans is domesticated in the culture medium containing high concentrations of Fe²⁺ ions.

The Bacillus coagulans strain is Bacillus coagulans iron-resistant Bacillus coagulans NT68 strain, which was preserved on Jan. 2, 2022 in the China General Microbiological Culture Collection Center, institute of Microbiology, Chinese Academy of Sciences, located at No. 3, Yard 1, Beichen West Road, Chaoyang District, Beijing, and the Preservation number is CGMCC No. 24223. The deposited strain was collected at the Institute of High Quality Waterfowl, Qingdao Agricultural University, 700, Changcheng Road, Chengyang District, Qingdao City, Shandong Province, P.R. China.

The application also provides a compound microbial agent, which includes domesticated Bacillus coagulans and Clostridium butyricum.

Optionally, the number ratio of Bacillus coagulans NT68 strain to Clostridium butyricum in the compound microbial agent is 1: 1-2.

According to the application, the Bacillus coagulans strain domesticated by high concentration of iron ions is co-cultured with Clostridium butyricum, so that the number of Bacillus coagulans is able to be increased, and the small peptide content in the domesticated Bacillus coagulans strain is able to be effectively increased. The microbial agent obtained by combining the Bacillus coagulans NT68 strain and Clostridium butyricum has good application effects, which is able to effectively enhance animal immunity, inhibit harmful bacteria, maintain intestinal health, promote growth and development, and improve the feed utilization rate.

### BRIEF DESCRIPTION OF THE DRAWINGS

FIG. 1A and FIG. 1B shows effect of domestication on a morphology of Bacillus coagulans.

FIG. 2 is a growth curve of Bacillus coagulans iron in a liquid medium containing Clostridium butyricum supernatant, in which Bacillus coagulans iron is fermented in common liquid medium without adding Clostridium butyricum (addition amount of 0%) in a control group, while Bacillus coagulans iron is fermented in a liquid medium containing Clostridium butyricum supernatant (with addition amount of 1%-5%) in experimental groups.

FIG. 3 is growth curve of Clostridium butyricum in a liquid culture medium containing fermentation supernatant of Bacillus coagulans, in which the Clostridium butyricum is fermented in a common liquid medium in a control group (with addition amount of fermentation supernatant of Bacillus coagulans of 0%), while Clostridium butyricum is fermented in a liquid culture medium containing different concentrations of fermentation supernatant of Bacillus coagulans in experimental groups.

FIG. 4 is a protein standard curve.

### DETAILED DESCRIPTION OF THE EMBODIMENTS

Bacillus coagulans is not a resident bacterium in the intestine, but a passing bacterium. It will not colonize or grow in the intestine for a long time. Bacillus coagulans will enter the gastrointestinal tract with food and eventually be excreted with feces, and play a role in the culture environment. Bacillus coagulans belongs to facultative anaerobic bacteria, which has high temperature resistance, strong gastric acid and bile salt resistance, and is able to maintain high vitality and inhibit the reproduction of harmful bacteria in the intestinal tract.

The Bacillus coagulans cultivated by intensive domestication is able to greatly enhance the tolerance of Fe²⁺ enrichment, and has good adaptability and bearing capacity for the enrichment of high-concentration Fe²⁺ ions. The domesticated bacteria have the characteristics of enriching iron, which lays a good foundation for producing iron-enriched Bacillus coagulans feed additives.

In the application, Bacillus coagulans and Clostridium butyricum are co-cultured, so that more organic irons are able to be enriched in Bacillus coagulans bacteria.

According to the present application, a specific composition of the culture medium is as follows: 5-15 g/L of peptone, 1-5 g/L of beef extract, 1-10 g/L of NaCl, Fe²⁺ with a concentration 250-350.0 mg/L, pH of 5.5-7.9±0.2. However, other culture media rich in iron ions may also be used, and vitamin C may be added to the media to prevent ferrous ions from being oxidized to ferric ions.

The present application will be described in detail with specific embodiments.

### Embodiment 1: Culture and Domestication of Bacillus coagulans

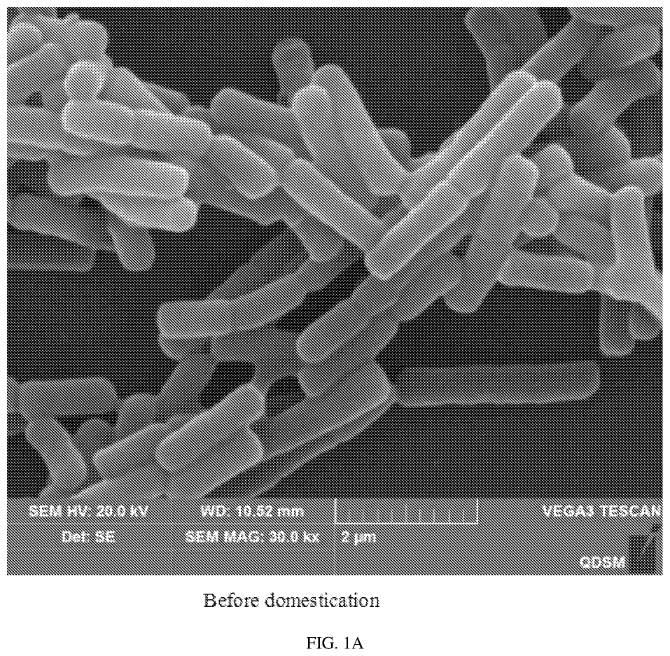

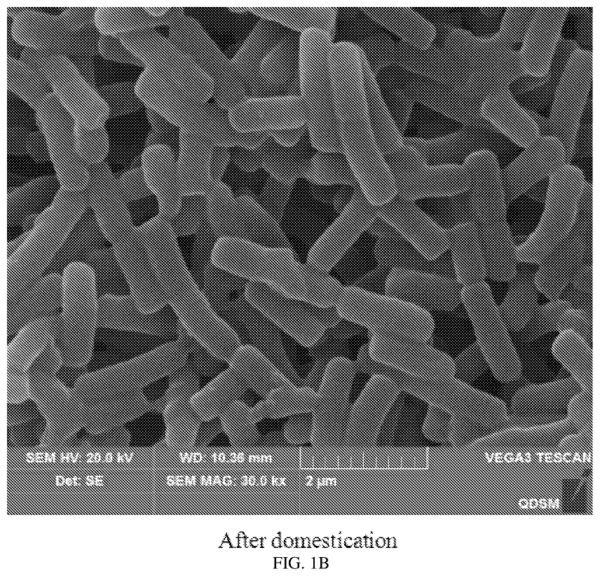

Domestication is carried out by gradually increasing the concentration of Fe²⁺ in the culture medium and increasing the culture temperature. After several generations of continuous culture, the domesticated strain is compared with Bacillus coagulans before domestication in terms of bacterial morphology, enzyme activity, small peptides and amino acids, etc. so as to further determine the properties and characteristics of Bacillus coagulans after domestication, and confirm the Bacillus coagulans after domestication is able to be stably multiplied for later generations. The effect of domestication on the morphology of Bacillus coagulans is shown in FIG. 1A and FIG. 1B. As can be seen from FIG. 1A and FIG. 1B, the bacterial surface texture of iron-enriched Bacillus coagulans becomes coarse. In order to resist the high concentration of iron ions, Bacillus coagulans changes its morphology, which shows that the appearance of Bacillus coagulans is changed obviously under the domestication of iron ions.

A domesticated strain of Bacillus Coagulans (Bacillus subtilis) NT68, which is able to tolerate high concentration of ferrous sulfate (300 mg/L), was preserved on Jan. 2, 2022 in the China General Microbiological Culture Collection Center, institute of Microbiology, Chinese Academy of Science, at No. 3, Yard 1, Beichen West Road, Chaoyang District, Beijing, with the preservation number of CGMCC No. 24223.

Common Bacillus coagulans and iron-enriched Bacillus coagulans (preservation number: CGMCC No. 24223) of the present application are respectively used for iron ion enrichment fermentation. The common Bacillus coagulans and iron-enriched Bacillus coagulans are inoculated in the culture medium containing ferrous ions with a concentration of 300 mg/L, respectively. After 24 hours of culture at 37° C., the bacterial liquid is centrifugally eluted and dried, and the bacterial content and organic iron content of the bacteria are determined.

The results show that when the iron content of is 300.0 mg/L, domesticated iron-enriched Bacillus coagulans has the bacterial content of 1.750 g/L, the organic iron content of the bacteria of 88.09 mg/kg, far greater than iron content of common Bacillus coagulans, which proves that the domesticated strain has good iron-enriched effects.

Moreover, because of the high temperature tolerance domestication in iron-enriched domestication, the copper-rich Bacillus coagulans screened and domesticated have better temperature tolerance than the conventional Bacillus coagulans that have not been domesticated. 40-50° C. is the suitable growth temperature of Bacillus coagulans. Under this temperature condition, Bacillus coagulans reproduces for one generation every 20-30 minutes. When the temperature exceeds 70° C., the growth rate of iron-enriched Bacillus coagulans is inhibited, but spores are able to be formed to resist the high temperature environment. However, the iron-enriched Bacillus coagulans domesticated by the application is able to survive at a high temperature above 90° C., and the survival rate is much higher than the survival rate of the common Bacillus coagulans strains that have not been domesticated. Yeast is not able to survive over 60° C., and Bifidobacterium is not able to survive over 70° C. The iron-enriched Bacillus coagulans screened and domesticated by the application is able to survive for about 10 minutes at 100° C. The results show that the domesticated iron-enriched Bacillus coagulans has obvious high-temperature resistance and the requirement that probiotics added in livestock and poultry pellet feed should have high-temperature resistance characteristics is further able to be satisfied.

### Embodiment 2: Symbiotic Effect of Bacillus coagulans NT68 Strain and Clostridium Butyricum

1. Growth promoting effect of cell-free supernatant of Bacillus coagulans NT68 strain and Clostridium butyricum

Clostridium butyricum used in the experiment is purchased from Shandong Sukehan Bioengineering Co., Ltd.

1) Preparation of cell-free supernatant of the two bacteria: a loop of strains is selected from the slant of the test tube of the two bacteria and inoculated into the corresponding 250 ml culture medium, with 6 replicates for each bacteria, and each bacteria is cultured for 24 hours at 37° C. (anaerobic culture of Clostridium butyricum), centrifuged at 6000 r/min for 10 minutes, and then autoclaved to obtain the cell-free supernatant of the two bacteria.

A loop of strains is selected from the slant of the test tube of the two bacteria and inoculated into the corresponding 250 ml culture medium, with 3 replicates for each bacteria, cultured for 24 hours at 37° C. (anaerobic culture of Clostridium butyricum), and the seed solutions of the two bacteria are obtained for counting separately through microscopy.

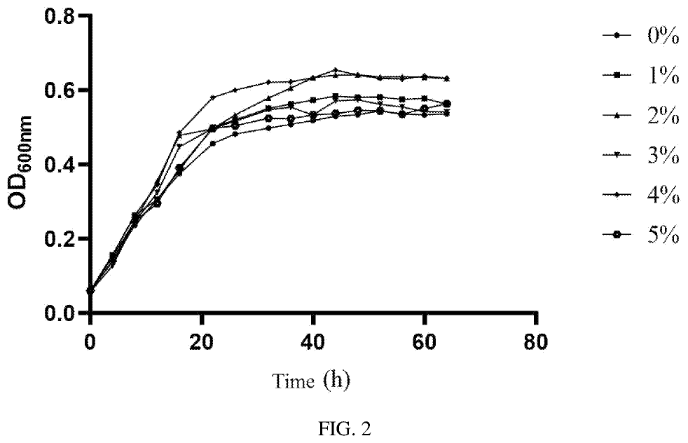

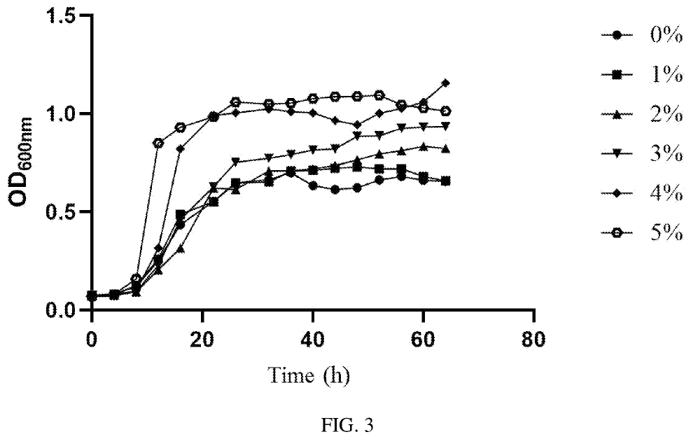

2) The symbiotic effect of two bacteria on bacterial growth: the fermentation broth is subjected to sampling every 4 hours to measure its optical density value at 600 nm (OD₆₀₀ₙₘ), so as to record the growth curves of Bacillus coagulans NT68 strain. The growth curves are shown in FIG. 2 and FIG. 3 respectively. FIG. 2 verifies the growth promoting effect of Clostridium butyricum fermentation products on Bacillus coagulans NT68 strain. The control group is Bacillus coagulans NT68 strain without Clostridium butyricum cell-free fermentation broth. The experimental groups are Bacillus coagulans NT68 strain with different amounts (1%, 2%, 3%, 4%, 5%) of Clostridium butyricum cell-free fermentation broth. The OD values of all experimental groups reaching the stable stage are higher than those of the control group without Clostridium butyricum cell-free fermentation broth, indicating that Clostridium butyricum fermentation products have a promoting effect on the growth of Bacillus coagulans. FIG. 3 verifies the promoting effect of the fermented products produced by Bacillus coagulans after domestication on the growth of Clostridium butyricum. The control group is Clostridium butyricum without Bacillus coagulans NT68 cell-free fermentation broth, while the experimental groups are Clostridium butyricum with different amounts (1%, 2%, 3%, 4%, 5%) of Bacillus coagulans NT68 cell-free fermentation broth. The OD values of all experimental groups reaching the stable stage are higher than that of the control group without Bacillus coagulans NT68 cell-free fermentation broth. The control group represents the growth of Clostridium butyricum without the supernatant of domesticated Bacillus coagulans.

The above results indicate that the domesticated Bacillus coagulans NT68 strain is able to form a synergistic effect with Clostridium butyricum, thereby promoting the growth of Bacillus coagulans NT68 strain. Meanwhile, Bacillus coagulans NT68 strain also has the ability to promote the growth of Clostridium butyricum.

The common Bacillus Coagulans is added to a liquid medium containing 300.0 mg/kg Fe²⁺ at a 3% inoculation size, and cultured in a constant temperature shaking incubator at 37° C. and 150 r/min for 24 hours before ending fermentation. the fermentation broth is centrifuged at 6000 r/min under 4° C. to obtain wet bacteria. The wet bacteria are rinsed with deionized water for 2-3 times, and the wet bacteria are dried at 55° C. to obtain Bacillus coagulans powder. A certain amount of Bacillus coagulans powder is weighed to measure the organic iron content of the bacteria. Under these conditions, the bacterial content is 0.93 g/L, and the organic iron content of the bacteria is 51.89 mg/kg. Compared with Bacillus coagulans co-cultured by domesticated Bacillus coagulans NT68 strain and Clostridium butyricum, the bacterial content of the common Bacillus coagulans is 46.86% lower and the iron content of bacteria is 41.09% lower.

2. The effect of synergistic effects on small peptide content

20 ml of liquid fermentation broth of two bacteria is taken, centrifuged for 10 minutes at 6000 r/min at 4° C. 5 ml of supernatant is taken, added with 10% Trichloroacetic acid of equal volume, and centrifuged for 10 minutes at 4000 r/min after standing for 0.5 hour to remove insoluble protein and long-chain peptide, and centrifuged for 5 minutes at 4000 r/min to take a certain amount of supernatant for Biuret method determination.

(1) Preparation method of reagent used in the experiment: standard protein solution: 0.4 g of bovine serum albumin is accurately weighed, dissolved in distilled water to a constant volume of 100 mL, and stored in a refrigerator at 4° C. Biuret Reagent: 1.50 g copper sulfate (CuSO₄:5H₂O) and 6.0 g potassium sodium tartrate (KNaC₄H₄O₆·4H₂O) are weighed, dissolved with 500 mL of water, added with 300 mL of 10% NaOH solution under stirring, diluted with water to 1 L, and stored in a plastic bottle. 10% Trichloroacetic acid (TCA): 1 mL of TCA is accurately weighed, and dissolved in distilled water to a constant volume of 10 mL.

(2) Calibration of protein standard curve: 1.0 g of sample is accurately weighed, added with 9 mL of water, mixed well, then added with 10 mL of 10% TCA solution, shaked and mixed well, let standing for 30 minutes, finally centrifuged, and the supernatant is taken for later use.

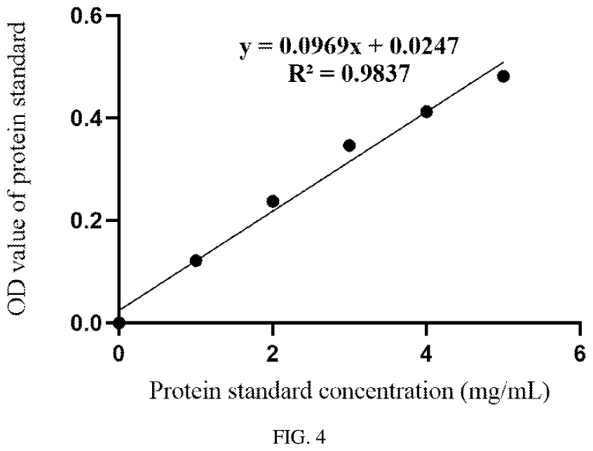

Above reagents are respectively added according to Table 1, mixed well, and reacted in dark for 30 minutes. After the reaction, the absorbance value at a wavelength of 540 nm is measured. Protein concentration is used as the x-axis and absorbance value as the y-axis of the standard curve. The standard curve results are shown in FIG. 4.

(3) Determination of small peptide content in fermentation broth: 1 mL of supernatant is accurately transferred into a test tube by using a pipette gun, added with 4 mL of distilled water, mixed well, added with 5 mL of Biuret reagent, mixed well, and reacted in dark for 30 minutes. After the reaction, the absorbance value is measure at a wavelength of 540 nm and the absorbance value is substituted into the standard curve to calculate the small peptide content of the sample. The drawing table of protein standard curve is shown in Table 1. The results of small peptide content are shown in Table 2.

TABLE 1
Drawing Table of Protein Standard Curve

| Reagent | 1 | 2 | 3 | 4 | 5 | 6 |
|---|---|---|---|---|---|---|
| Protein standard solution (ml) | 0 | 1 | 2 | 3 | 4 | 5 |
| Distilled water (ml) | 5 | 4 | 3 | 2 | 1 | 0 |
| Biuret reagent (ml) | 5 | 5 | 5 | 5 | 5 | 5 |

TABLE 2
Effect of the symbiotic effect of Bacillus coagulans iron
and Clostridium butyricum on small peptide content

| | | | | | | BC supernatant promoting CB | | |
|---|---|---|---|---|---|---|---|---|
| Items | | | | | | fermentation test | | |
| | | BC + CB | CB supernatant promoting BC | | | CB + BC | | |
| | | fermentation | fermentation test | | | fermentation | | |
| | BC | broth | SEM | P | CB | broth | SEM | P |
| Content of | 1.07^{b} | 1.29^{a} | 0.04 | <0.01 | 0.49^{b} | 0.89^{a} | 0.07 | <0.01 |
| small | | | | | | | | |
| peptides | | | | | | | | |
| (mg/mL) | | | | | | | | |

Note:
BC represents the domesticated Bacillus coagulans NT68 strain;
CB is Clostridium butyricum

The experiment on the effect of CB supernatant on BC fermentation proves that the addition of Clostridium butyricum fermentation broth is able to improve the ability of Bacillus coagulans NT strain to produce small peptides. The BC group shows the content of small peptides produced by fermentation broth Bacillus coagulans NT68 strain cultured in common medium, while the BC+CB supernatant group shows the content of small peptides produced by fermentation broth of Bacillus coagulans NT68 strain cultured in the culture medium with Clostridium butyricum fermentation broth. The experimental results show that under the influence of Clostridium butyricum fermentation broth, the small peptide content produced by Bacillus coagulans NT68 strain significantly is increased (p<0.01).

The experiment on the effect of BC supernatant on CB fermentation confirms that the addition of fermentation broth of Bacillus coagulans NT68 strain is able to improve the ability of Clostridium butyricum to produce small peptides. The CB group shows the content of small peptides produced by the Clostridium butyricum fermentation broth cultured in common culture medium, while the CB+BC supernatant group shows the content of small peptides produced by the Clostridium butyricum fermentation broth cultured in the culture medium with fermentation broth of Bacillus coagulans NT68 strain. The experimental results show that the content of small peptides produced by the Clostridium butyricum is significantly increased under the influence of the fermentation broth of Bacillus coagulans NT68 strain (p<0.01).

The above results indicate that Bacillus coagulans NT68 strain has a synergistic interaction effect with Clostridium butyricum.

### Embodiment 3: Iron-Enriched Bacillus coagulans NT68 Strain and Clostridium Butyricum Synergistically Inhibit the Growth and Reproduction of Harmful Intestinal Bacteria

(1) Activation of bacterial strains: sterile inoculation is carried out on an ultra-clean workbench. 0.1 ml of Escherichia coli is inoculated onto LB (Luria-Bertani) agar medium. 0.1 ml of Salmonella is inoculated onto NA (Nutrient Agar) medium. 0.1 ml of Bacillus coagulans NT68 strain and 0.1 ml of Clostridium butyricum are inoculated onto NA medium, respectively. They are cultured in a constant temperature incubator at 37° C. for 24 hours and placed at 0-4° C. for later use.

(2) Preparation of bacterial suspensions of intestinal harmful bacteria: on the ultra-clean workbench, Escherichia coli is picked from the slant of activated bacteria and inoculated in LB broth medium suitable for growth, and Salmonella is picked and inoculated in NB nutrient broth medium for constant temperature culture. After 24 hours of cultivation at 37° C., microscopic counting is carried out to prepare bacterial suspensions of 1.0×10⁶ CFU/mL, which is fully shaken.

(3) Preparation of fermentation broths of iron-enriched Bacillus coagulans NT68 strain and Clostridium butyricum: on the ultra-clean workbench, Bacillus coagulans NT68 strain is selected from the slant of the activated bacteria and inoculated into NA nutrient broth culture medium. It is cultured for 24 hours at 180 r/min in a 45° C. shaker, and Clostridium butyricum is picked and inoculated into RCM (Reinforced Clostridium Medium) culture medium. It is cultured or 24 hours at 180 r/min in a 45° C. shaker. The cultured bacterial suspensions are centrifuged to obtain the supernatants, which are the fermentation broths.

(4) Preparation of zone of inhibition: intestinal harmful bacteria are evenly spread and inoculated on the surface of the agar plate, punching 6 holes with a hole diameter of 8 is performed by a sterilized metal punch. The distilled water, single Bacillus coagulans iron fermentation broth, single Clostridium butyricum fermentation broth, 1:1 mixed fermentation broth, 1:2 mixed fermentation broth, 2:1 mixed fermentation broth are respectively added, with 0.2 ml of above liquid added to each hole. Incubation is performed at 37° C. for 24-48 hours, and then the diameter of zone of inhibition is measured with Vernier scale to judge the joint bacteriostatic effect of Bacillus coagulans iron and Clostridium butyricum as shown in table 3 below.

TABLE 3
diameter of zone of inhibition of Bacillus coagulans iron and
Clostridium butyricum fermentation broth against harmful bacteria

| | Diameter of zone of inhibition | |
|---|---|---|
| Liquid | Escherichia coli | Salmonella |
| Distilled water | 8 | 8 |
| Single Bacillus coagulans | 14.24 | 14.04 |
| iron fermentation broth | | |
| Single Clostridium butyricum | 12.86 | 13.66 |
| fermentation broth | | |
| 1:1 mixed fermentation broth | 18.92 | 17.38 |
| 1:2 mixed fermentation broth | 17.14 | 16.54 |
| 2:1 mixed fermentation broth | 16.22 | 17.26 |

The diameter of the zone of inhibition produced by the mixed fermentation broth is greater than the diameter of the zone of inhibition produced by the single bacterial fermentation broth, indicating that the synergistic inhibition ability of Bacillus coagulans NT68 strain and Clostridium butyricum is greater than inhibition ability of a single bacterium. Among them, when Bacillus coagulans: Clostridium butyricum fermentation broth is 1:1, the zone of inhibition is the largest and the inhibition effect is the strongest.

The above results indicate that Bacillus coagulans NT68 strain and Clostridium butyricum are able to synergistically inhibit the growth and reproduction of Escherichia coli.

### Embodiment 4: Effect of Bacillus coagulans NT68 Strain on the Growth and Development of Meat Ducks

In this experiment, 180 7-day-old male Beijing Cherry Valley duck ducklings are selected and fed adaptively for 5 days. The ducklings are randomly divided into 6 groups based on body weights, with 5 replicates per group and 6 ducklings per replicate. This experiment involves 5 treatment groups and 1 control group. NP group: blank control group, fed with basic diet (the composition of the basic diet is shown in Table 4); BC group: Bacillus coagulans NT68 strain domesticated with iron is added to the basic diet; CB group: Clostridium butyricum is added to the basic diet; BC+CB (1:1) group: a mixed probiotic preparation is added on the basis of the basic diet (iron-domesticated Bacillus coagulans NT66 strain and Clostridium butyricum are added in a 1:1 mixture); BC+CB (1:2) group: a mixed probiotic preparation was added on the basis of the basic diet (iron domesticated Bacillus coagulans NT68 strain and Clostridium butyricum were added in a 1:2 mixture); BC+CB (2:1) group: on the basis of the basic diet, a mixed probiotic preparation is added (iron-domesticated Bacillus coagulans NT68 strain and Clostridium butyricum are added in a 2:1 mixture). The added probiotic preparations ensure a bacterial concentration of 1×10⁸ CFU/ml (colony-Forming Units per milliliter). See Table 5 for design of experiment. The feeding cycle is 6 weeks, and after the end of the 6th week, slaughter sampling will be conducted.

TABLE 4
Composition of Basic Diet for Meat Ducks

| | Content | Nutrient | |
|---|---|---|---|
| Raw material | (%) | composition ² | Content |
| Corn | 63.55 | Metabolic energy | 3131.86 |
| | | (MJ/kg) | |
| Soybean meal | 23.85 | Crude protein % | 17.0 |
| Soybean oil | 5.0 | Crude fiber (%) | 4.31 |
| Chrysanthemum stem | 3.24 | Crude fat (%) | 8.17 |
| Calcium hydrogen phosphate | 1.59 | | |
| Stone powder | 1.23 | | |
| Salt | 0.3 | | |
| L-lysine monohydrochloride | 0.06 | | |
| Tryptophan | 0.01 | | |
| Premix ¹ | 1 | | |
| DL-Methionine (99%) | 0.17 | | |
| Total | 100 | | |

¹ The premix provides the following micronutrient (per kilogram of whole food): VA (Vitamin A) 12 000 IU, VD₃ 2 500 IU, VE 20 mg, VK₃ 3 mg, VB₁ 3 mg, VB₂ 8 mg, VB₆ 7 mg, VB₁₂ 0.03 mg, D-pantothenic acid 20 mg, nicotinic acid 50 mg, folic acid 1.5 mg, biotin 0.1 mg, choline 500 mg, copper (as Copper sulfate) 9 mg, zinc (as zinc sulfate) 110 mg, iron (as Iron(II) sulfate) 100 mg, iron (as Iron(III) sulfate) 100 mg, selenium (as sodium selenite) 0.16 mg, iodine (as potassium iodide) 0.6 mg.
² Both nutrient content are calculated values

TABLE 5
Design of experiment of Meat Duck Feeding

| Items | Design of experiment |
|---|---|
| NP group | Basic diet (BD) |
| (control group) | |
| BC group | BD + 1 × 10⁸ CFU/mL (BC) |
| BC + CB (1:1) group | BD + 0.5 × 10⁸ CFU/mL BC + |
| | 0.5 × 10⁸ CFU/mL CB |
| BC + CB (1:2) group | BD + 0.33 × 10⁸ CFU/mL BC + |
| | 0.66 × 10⁸ CFU/mL CB |
| BC + CB (2:1) group | BD + 0.66 × 10⁸ CFU/mL BC + |
| | 0.33 × 10⁸ CFU/mL CB |

The experimental results show that at the age of 35 days, the average daily gain of meat ducks whether the meat ducks are fed with Bacillus coagulans iron NT68 strain or the meat ducks are feed with Bacillus coagulans NT68 strain in combination with Clostridium butyricum is significantly higher than that of the control group (p<0.05). For the average daily feed intake, there was no significant difference (p>0.05), but the control group has a higher average daily feed intake than the other experimental groups. In addition, adding Bacillus coagulans NT68 strain or a mixture of Bacillus coagulans NT68 strain and Clostridium butyricum to the feed significantly reduces the ratio of feed to gain (F/G) (p<0.01), indicating that domesticated iron ion-enriched Bacillus coagulans is able to improve the feed conversion rate of meat ducks (Table 6).

TABLE 6
Growth Performance of 35-Day-Old Meat Ducks

| | Average daily | Average daily | |
|---|---|---|---|
| Items | gain/kg | feed intake/kg | F/G |
| Control group (NP) | 0.40^{b} ± 0.01 | 1.03^{ab} ± 0.02 | 2.56^{a} ± 0.01 |
| BC group | 0.41^{b} ± 0.01 | 0.98^{ab} ± 0.03 | 2.40^{b} ± 0.04 |
| BC + CB (1:1) group | 0.42^{ab} ± 0.01 | 0.99^{ab} ± 0.03 | 2.36^{b} ± 0.07 |
| BC + CB (1:2) group | 0.44ª ± 0.01 | 1.00ª ± 0.02 | 2.35^{b} ± 0.05 |
| BC + CB (2:1) group | 0.42ª^{b} ± 0.01 | 0.96^{b} ± 0.02 | 2.26^{b} ± 0.03 |

In summary, the method of gradually increasing the concentration of Fe²⁺ in the culture medium and culture temperature adopted by the present application for domestication and screening of Bacillus coagulans NT68 strain not only possesses the advantages of probiotics of Bacillus coagulans, but also reduces the oxidation of ferrous ions (Fe²⁺), improves the ability of inorganic iron to convert into organic iron of the bacteria, greatly improves the utilization rate of trace elements and feed conversion rate of livestock and poultry, promotes growth and development, and enhances the quality of livestock and poultry products, better utilizes the biological functions of organic iron.

What is claimed is:

1. A compound microbial agent, comprising Bacillus coagulans and Clostridium butyricum,wherein a preservation number of the Bacillus coagulans is CGMCC No. 24223, and a number ratio of Bacillus coagulans strains to the Clostridium butyricum is 1: 1-2.
